(19) United States
(12) Patent Application Publication
(10) Pub. No.: US 2014/0051139 A1
(43) Pub. Date: Feb. 20, 2014

(54) PROCESSES FOR BIOCONVERSION OF SYNGAS TO OXYGENATED ORGANIC COMPOUND PROVIDING STORAGE AND REACTIVATION OF MICROORGANISM CONCENTRATES

(71) Applicant: Coskata, Inc., Warrenville, IL (US)

(72) Inventor: Peter Martin Lokken, Chicago, IL (US)

(73) Assignee: Coskata, Inc., Warrenville, IL (US)

(21) Appl. No.: 13/970,010

(22) Filed: Aug. 19, 2013

Related U.S. Application Data
(60) Provisional application No. 61/691,052, filed on Aug. 20, 2012.

(51) Int. Cl. C12P 7/06

(52) U.S. Cl. CPC C12P 7/065
USPC 435/140; 435/161; 435/160; 435/157; 435/154

(57) ABSTRACT

Processes are disclosed for preparing microorganism concentrates from fermentation broth containing a free suspension of the microorganisms which is used for the anaerobic conversion of syngas to oxygenated organic compound. The processes involve the use of processing steps and the presence of certain additives to enhance the ability of the microorganism concentrate to be stored for extended periods and reactivated.

### CROSS REFERENCE TO RELATED APPLICATIONS

Priority is claimed to U.S. Provisional Application No. 61/691,052, filed Aug. 20, 2012, which is hereby incorporated by reference in its entirety.

### FIELD OF THE INVENTION

This invention pertains to processes for the anaerobic bioconversion of syngas to oxygenated organic compound, particularly to processes where microorganisms are recovered and stored as a concentrate for use to replenish the population of microorganism in a reactor or to start-up a reactor. The invention also pertains to process for the storage of such microorganism concentrates and the reactivation of the stored microorganism concentrates.

### BACKGROUND

Oxygenated organic compounds such as ethanol, acetic acid, propanol and butanol are currently commercially produced in large scale facilities through petrochemical and metabolic processes. For instance, in the United States over 10 billion gallons of ethanol are produced from corn sugars through fermentation processes to achieve favorable economics for use of the ethanol as a biofuel.

Recently efforts have been devoted to produce oxygenated organic compound using feedstocks other than fossil fuels and biomass that can be used as food or animal feed. One such proposal is the use of anaerobic fermentations of carbon monoxide and of hydrogen and carbon dioxide to produce oxygenated organic compound. For such processes to be commercially viable they must be able to benefit from the advantages of scale, and thus facilities using these processes need to be able to produce upwards of 50 or 100 million or more gallons of oxygenated organic compound per year.

These anaerobic fermentation processes necessarily involve the mass transfer of substrate from the gas phase into the liquid phase for access by the microorganisms. These mass transfer considerations together with economies of scale, tend to favor the use of large reactors for commercial-scale facilities. The start-up of these facilities can be problematic due to the large volume of microorganisms required and the time required to grow a sufficient population of the microorganisms. The period of time required to start up a commercial scale reactor, which may have a volume of 5 million liters or more, may be a week or more. Additionally, any disruption in the supply of substrate or contamination from other microorganisms, mutations or phages, can result in the need to shut down and freshly re-start a reactor.

The microorganisms for the anaerobic fermentation typically are expected to be generated by seed farms at the site of the facility. Usually the seed farms are comprised of a sequential series of reactors of increasing size with the final reactor having enough volume to provide an initial charge to the commercial-scale reactor. Usually, the growth in each seed farm stage is targeted to increase the size of the population by a factor of 10 and each stage usually takes from 2 to 7 days to achieve the sought growth. Once charged from the seed farm, the reactor is then operated to promote the growth of the population of microorganisms while increasing the volume of the aqueous medium in the reactor until steady-state is achieved. The capital and operating expense for a seed farm is not insignificant.

R. Hickey, in U.S. Published Patent Application No. 2013/0078693 A1, hereby incorporated by reference in its entirety, discloses processes for the start-up of commercial-scale anaerobic fermentation reactors for the bioconversion of carbon monoxide and hydrogen to oxygenated organic product. The disclosed processes modulate the mass transfer of carbon monoxide to the aqueous fermentation medium to provide for rapid growth of the population of microorganisms while avoiding carbon monoxide inhibition.

Accordingly, processes are sought to obtain microorganisms from the fermentation reactor and store the microorganisms until needed, and to do so in a cost effective manner in which the viability of the microorganisms is retained and in which the processes for obtaining and storing the microorganisms does not cause undue stress on the microorganisms such that mutations or other changes occur.

Common practice for storing microorganisms for long periods of time is freeze drying where the metabolic rate of the microorganisms for all practical purposes ceases. The freeze dried microorganisms can then be reactivated in a fermentation broth. Freeze drying and storage, particularly for the large volumes of microorganisms required for a commercial scale reactor, is commercially impractical.

Another common practice, especially for laboratory scale work, is to cool the fermentation broth and store it at refrigerator temperature (about 4° C.). Abrini, et al., in “Clostridium autoethanogenum, ps. Nov., an anaerobic bacterium that produces ethanol form carbon monoxide”, Arch Microbio. (1994) 161:345-351, demonstrate the effect of temperature on the metabolic activity of Clostridia. At 20° C. the growth rate is less than about 20 percent of that at the optimal 37° C. The cooling reduces the metabolic activity of the microorganism to enable storage without the addition of substrate. Cooling large volumes of fermentation broth as would exist in commercial-scale operations to these refrigeration temperatures is problematic. Substantial energy is required to reduce the temperature of the fermentation broth and to do the cooling rapidly to avoid death of the microorganisms. Further, large storage facilities capable of maintaining the fermentation broth at refrigeration temperatures would be required. Additionally, transportation of the fermentation broth would require refrigerated vessels.

The feasibility of maintaining a fermentation broth viable would be enhanced if storage can occur at warmer temperatures. Several proposals exist to maintain microorganisms viable at warmer temperatures. In general, these processes involve adding substrate to the microorganisms, and reducing the amount of substrate added by maintaining the microorganisms at temperatures below the optimum temperatures, i.e., cooling to reduce metabolic rate.

For instance, Adams, et al., in United States Published Patent Application No. 2010/0227377 A1 disclose a method for sustaining microorganism culture in syngas fermentation by adding carbon dioxide and optionally alcohol, such as ethanol, to the reactor in the event of a disruption of syngas feed. The applicants recite that the temperature of the reactor should be decreased, e.g., to 0° C. to 25° C., certain free acetic acid concentrations be maintained and these steps being performed within a specified time.

Adams, et al., do not provide any disclose or suggest any method for providing under normal operation an inventory of microorganisms for replenishment or restart of a commercial-scale bioreactor nor any method for maintaining the viability of an inventory of microorganisms in a static system, i.e., a system having the absence of addition of substrate or other nutrients.

In another proposal, Simpson, et al., in U.S. Published Patent Application No. 2011/0281336 A1 disclose methods of sustaining culture viability of carboxydotrophic bacteria during periods of limited substrate supply by maintaining the temperature of the microbial culture at a temperature below an optimum operating temperature. The applicants are concerned with disruption of the supply of carbon monoxide from, e.g., a steel mill operation. It appears that the fermentation broth is cooled although the applicants state that the culture can be transferred from the bioreactor to a storage vessel and/or transport vessel. The process thus is useful for temporary outages or for storage or transport of the fermentation broth.

Simpson, et al., appear to indicate a storage of the microorganism for relatively short periods of time. At paragraph 0046 they state:
“In accordance with the methods of this invention, it has been surprisingly recognized that carboxydotrophic microbial cultures may be stored with minimal or no additional substrate feeding and/or agitation, at temperatures below their optimum.”

The example provided by Simpson, et al., is instructive. They transfer 50 ml. of active culture to a sterile serum bottle and then fill the head space (184 ml.) with carbon monoxide-containing gas at a pressure of 40 psia. Serum bottles are then stored at a set temperatures for various periods of time (3, 6, 24 and 31 hours). At 31 hours and storage at 4° C. (refrigeration temperature), the stored culture is reported to be as viable as it is after only 3 hours of storage. Warmer temperatures are reported to shorten the duration of storage that the active culture can be maintained. At 37° C., the culture is reported to be not viable after 3 hours of storage. At 24° C., 6 hours of storage reduced viability and the culture was reported not to be viable at 24 hours of storage. At 14° C., the culture had reduced viability upon 31 hours of storage.

Thus, Simpson, et al., do not disclose or suggest alternative, viable methods to refrigeration (4° C.) for long term storage of Clostridia, even in the presence of substrate in the head space. In this regard, Simpson, et al., in paragraph 0112, attribute the lack of survival of the microorganisms to the low mass transfer of substrate in the head space to the fermentation broth.
“It is considered that without agitation, the active microbial culture rapidly depletes the limited CO dissolved in the liquid nutrient medium. The excess carbon monoxide in the headspace may have limited transfer into the liquid nutrient medium. However, in the absence of agitation, it is expected there will be a CO gradient, wherein the uppermost surface of the liquid nutrient medium may have a relatively high CO concentration, but this will decrease down through the medium. In the absence of agitation, the microbial cells will settle to the bottom of the vial, where they will be substantially starved of substrate and will rapidly decrease in viability. Subsequently, the deteriorated or dead culture is unsuitable for inoculation.”

Simpson, et al., appear to imply that more concentrated microbial cultures increase the problem of carbon monoxide depletion and mass transfer to the cultures. At paragraph 0092, Simpson, et al., state:
“In particular embodiments of the invention, the stored microbial culture is used for inoculation of a bioreactor. In such embodiments, it is desirable that the culture is suitably dense (i.e. large number of microbes per unit volume) and that the viability of the culture is substantially sustained during storage (i.e. transport to a remote location). Typically the higher density of the microbial cells in the culture, the faster they will deplete any CO available in the liquid nutrient medium. Without wishing to be bound by theory, it is considered that when CO is not available, or is sufficiently depleted, the viability of the microbial culture decreases. For example, at least a portion of the culture begins to die off and/or the culture switches to a slower metabolism, such that when a bioreactor is inoculated with the microbial culture, there is a lag before high growth rates and/or productivity is attained. However, when the culture is cooled, the depletion of CO in the liquid nutrient medium is slowed such that culture viability is substantially preserved over an extended period.”
   Simpson, et al., do not disclose or suggest any process where the bioreactor can effectively be used to provide under normal operation an inventory of microorganisms for replenishment or restart of a commercial-scale bioreactor.

Lewis, et al., in U.S. Published Patent Application No. 2007/0275447 discuss a novel Clostridium carboxidivorans bacteria referenced by them as “P-7”. Lewis, et al., state at paragraphs 0059 and 0060:
“Once purified, P7 was maintained as a biologically pure culture in the laboratory under the following conditions: P7 was routinely maintained by transferring into fresh medium every 1-2 weeks. Cultures can, however, be stored on the bench for over a year. For longer term storage, cultures can be lyophilized and frozen, or stored in 50% glycerol at −20° C. Such techniques for the storage and handling of anaerobic bacteria are described, for example, in Sower and Schreier (1995, Archea, A Laboratory Manual, Methanogens, Cold Spring Harbor Press).
During the culture and storage of P7, it was observed that this organism displayed exceptionally stability, robustness, and flexibility. For example, as noted above, cultures are stable on the bench at room temperature for extended periods of time. Cultures of P7 can recover from an exposure to 2% oxygen in the gas phase and continue to produce ethanol from carbon monoxide during recovery. P7 cultures exhibited the ability to resume initial performance following major changes in selected critical operating parameters (e.g. pH, temperature, etc.). In addition, cultures of P7 reach a cell density of 1 O.D. units in a short period of time (e.g. about 24 hours) and the P7 culture does not readily lyse out.”

D. R. Boone, in Protocol 7: Short- and Long-term Maintenance of Methanogen Stock Cultures”, in Sower and Schreier, 1995, Archea, A Laboratory Manual, Methanogens, Cold Spring Harbor Press, pages 79 to 83, indicates that many methanogens die rapidly, with no viable cells remaining in cultures after a few hours to several days. He states that: “Cultures catabolizing H₂ plus CO₂ survive longer when they are repressurized with H₂ plus CO₂ and stored at a temperature below their optimal growth temperature (at room temperature or in a refrigerator).” Hence, Sower, et al., do not provide any insights as to the procedures used by Lewis, et al., to obtain bench stability of P7 other than that repressurization with H₂ plus CO₂ is likely required. Simpson, et al., as stated above, express concerns that the mass transfer of hydrogen into the culture would not be sufficient to accommodate the needs of the microorganisms. The mass transfer issues would likely not exist with cultures containing a very low density of cells or with storage techniques that provide a high surface area per unit volume, e.g., a thin liquid layer, neither of which would be viable for storage of commercial volumes of microorganisms.

J. Du, et al., in U.S. Published Patent Application No. 2013/0137151 A1, hereby incorporated by reference in its entirety, disclose a process for the producing oxygenated organic compound from syngas in which the aqueous fermentation medium is withdrawn from a bioreactor and subjected to centrifugation to provide a liquid stream relatively free of solids that can be passed to a distillation assembly to recover the oxygenated organic compound and a solids-containing stream that can be recycled to the bioreactor.

A desire exists for processes that can collect and store for extended periods of time microorganisms from bioreactors without adversely affecting the operation of the bioreactor to produce oxygenated organic compound. It is further desired that such processes can be implemented with low capital and operating costs and preferably in a static environment.

### SUMMARY OF THE INVENTION

In accordance with this invention, processes are provided for the accumulation and storage of microorganisms from commercial-scale anaerobic fermentation bioreactors for the production of oxygenated organic compound. The processes can be used without adverse effect on the productivity of the bioreactor to produce oxygenated organic compound. Advantageously, the processes are integrated with the unit operations for the recovery of the oxygenated organic compound from the fermentation broth. Moreover, the preferred processes of this invention provide for a static storage of the microorganisms.

The processes of this invention can reduce stress on the microorganisms during formation of microbial, or microorganism, concentrates, storage of the concentrates and reactivation of the microorganisms from the stored microorganism concentrate. The reduced stress can attenuate the risk of losing cultural performance consistency.

The preferred process of this invention enable a high retention of metabolic activity from the microorganism concentrate, even when stored at 10° C. to 20° C. under static conditions.

One broad aspect of this invention pertains to integrated processes for the anaerobic bioconversion of a gas feed containing gas substrate comprising at least one of carbon monoxide and of carbon dioxide and hydrogen in a fermentation broth containing a suspension of microorganisms suitable for converting said gas substrate to oxygenated organic compound and for providing a microorganism concentrate comprising:
a. contacting the gas feed with the fermentation broth under fermentation conditions in a bioreactor to bioconvert at least a portion of the gas substrate to oxygenated organic compound;
b. continuously or intermittently withdrawing from the bioreactor a portion of the fermentation broth to maintain the concentration of the oxygenated organic compound below that which would unduly adversely affect the microorganisms, said withdrawn portion containing microorganisms;
c. separating at least an aliquot portion of the withdrawn fermentation broth to provide a solids depleted liquor and a solids rich stream;
d. recovering a product comprising said oxygenated organic solvent from the solids depleted liquor;
e. diluting at least a portion of the solids rich stream with a cool, diluting water stream to provide a combined stream having a temperature below about 25° C. sufficient to reduce the metabolic activity of the microorganisms;
g. separating the combined stream into a microorganism concentrate having a solids content of at least about 30, preferably at least about 70, grams per liter and a solids depleted water stream;
h. storing, preferably statically, said microorganism concentrate at a temperature below about 25° C.

Advantageously, the diluting water stream is used for direct heat exchange to cool the solids rich stream quickly and without undue stress on the microorganisms. The cooling reduces the metabolic activity of the microorganisms. Step (c) results in a solids rich stream having substantial reduction of nutrients and oxygenated organic product per unit mass of microorganisms as compared to that of the withdrawn fermentation broth. Preferably nutrients are provided to the microorganism concentrate, and the diluting water stream may be used to provide all or a portion of these nutrients. These nutrients can include carbon sources such as lower alkanols, sugar-based moieties, citrate and the like that may be metabolized by the microorganisms during storage and/or during reactivation. The presence of sulfur source and reductant such as cysteine and other additives can also enhance the viability of the microorganism concentrate during storage and reactivation. If desired, the diluting water stream may also be adapted to flush out oxygenated organic compound from the solids rich stream and thus provide a lower concentration of oxygenated organic compound in the microorganism concentrate.

A second broad aspect of the invention relates to an improvement in processes for providing a microorganism concentrate from a fermentation broth containing a free suspension of said microorganism from an anaerobic bioconversion of substrate comprising at least one of carbon monoxide and of carbon dioxide and hydrogen to oxygenated organic compound by separation to provide a solids depleted stream and a microorganism concentrate, wherein the improvement comprises providing a concentrate containing at least about 70, preferably at least about 100, say between about 100 and 250, grams of solids per liter. At these high concentrations, a suppression of the metabolic activity of the population of microorganisms is facilitated.

A third broad aspect of the invention relates to an improvement for providing a microorganism concentrate from a fermentation broth containing a free suspension of said microorganism from an anaerobic bioconversion of substrate comprising at least one of carbon monoxide and of carbon dioxide and hydrogen to oxygenated organic compound by separation to provide a solids depleted stream and a microorganism concentrate, wherein the improvement comprises providing a carbon source to the concentrate such as one or more of sugar-based moieties and citrate.

A fourth broad aspect of the invention relates to an improvement for storing a microorganism concentrate of microorganisms for anaerobic bioconversion of substrate comprising at least one of carbon monoxide and of carbon dioxide and hydrogen to oxygenated organic compound, wherein the improvement comprises providing carbon source in said microorganism concentrate. Advantageously, the improvement further comprises maintaining a partial pressure of carbon dioxide above the microorganism concentrate of at least about 20 kPa to modulate metabolic rates and to facilitate reactivation.

A fifth broad aspect of the invention pertains to concentrating microorganisms from a fermentation broth containing a free suspension of said microorganism used in an anaerobic bioconversion of substrate comprising at least one of carbon monoxide and of carbon dioxide and hydrogen to oxygenated organic compound to provide a highly viscous microorganism concentrate that reduces intermixing of concentrate during storage such that the inventory of microorganism concentrate can be administered on a first in, first out basis to maintain a desired average storage age of the microorganisms.

A sixth broad aspect of the invention pertains to processes for reactivating a microorganism concentrate containing microorganism for anaerobic bioconversion of substrate comprising at least one of carbon monoxide and of carbon dioxide and hydrogen to oxygenated organic compound that has been stored under reduced metabolic conditions, wherein the improvement comprises providing carbon source such as sugar-based moiety and citrate in said microorganism concentrate.

### BRIEF DESCRIPTION OF THE DRAWING

FIG. 1 is a schematic depiction of an apparatus for practicing the processes of this invention.

### DETAILED DISCUSSION

### Definitions

Oxygenated organic compound means one or more organic compounds containing two to six carbon atoms selected from the group of aliphatic carboxylic acids and salts, alkanols and alkoxide salts, and aldehydes. Often oxygenated organic compound is a mixture of organic compounds produced by the microorganisms contained in the fermentation broth.

Fermentation broth means a liquid water phase which may contain dissolved compounds including, but not limited to hydrogen, carbon monoxide, and carbon dioxide.

Biomass means biological material of living or recently living plants and animals and contains at least hydrogen, oxygen and carbon. Biomass typically also contains nitrogen, phosphorus, sulfur, sodium and potassium. The chemical composition of biomass can vary from source to source and even within a source. Sources of biomass include, but are not limited to, harvested plants such as wood, grass clippings and yard waste, switchgrass, corn (including corn stover), hemp, sorghum, sugarcane (including bagas), and the like; and waste such as garbage and municipal waste. Biomass does not include fossil fuels such as coal, natural gas, and petroleum.

Microorganism concentrate is an aqueous slurry containing at least about 30 grams of solids (cells and solid cell debris) per liter.

Reactivation means reestablishing metabolic activity of a population of microorganisms that have been in a stasis or significantly reduced metabolic state.

For a syngas to oxygenated organic compound fermentation process to be commercially viable, capital and operating costs must be sufficiently low that it is at least competitive with alternative processes to make oxygenated organic compound. For instance, ethanol is commercially produced from corn in facilities having name plate capacities of over 100 million gallons per year. Accordingly, the syngas to oxygenated organic compound fermentation process must be able to take advantage of similar economies of scale. Thus, a commercial scale facility may require at least 5, preferably at least 20, million liters of fermentation reactor capacity.

The term syngas will be used herein to mean any gas containing at least one of carbon monoxide and hydrogen, and preferably including the presence of carbon dioxide, and will thus include gas substrates other than conventional synthesis gas containing both carbon monoxide and hydrogen.

The term sugar-based moieties as used herein will mean sugars and sugar alcohols of 5 to 12 carbons. Sugar alcohols include, but are not limited to xylitol and sorbitol.

The term static (statically) means that substantially no fluid (gas or liquid) or solid is distributed within the microorganism concentrate. Distributed within means that the fluid or solid is actively introduced into the interior of the microorganism concentrate. Static storage conditions exist where a quiescent microorganism concentrate is maintained below a gas contained in the headspace.

The use of the terms “a” and “an” is intended to include one or more of the element described, and unless explicit or otherwise clear from the context, an element recited in the singular is intended to include one or more of such elements.

### a. Overview

The processes of this invention pertain to the conversion of biomass to oxygenated organic compound by gasification to provide a substrate containing carbon monoxide, hydrogen and carbon dioxide and bioconversion of the substrate to the oxygenated organic compound via anaerobic fermentation.

### Substrate and Gas Feed

Anaerobic fermentation to produce oxygenated organic compound uses a substrate comprising carbon monoxide and/or carbon dioxide and hydrogen, the latter being for the hydrogen conversion pathway. The gas feed will typically contain nitrogen and methane in addition to carbon monoxide and hydrogen. Syngas is one source of a gas substrate. Syngas can be made from many carbonaceous feedstocks. These include sources of hydrocarbons such as natural gas, biogas, biomass, especially woody biomass, gas generated by reforming hydrocarbon-containing materials, peat, petroleum coke, coal, waste material such as debris from construction and demolition, municipal solid waste, and landfill gas. Syngas is typically produced by a gasifier, reforming or partial oxidation. The syngas produced thereby will typically contain from 10 to 60 mole % CO, from 10 to 25 mole % CO₂ and from 10 to 60 mole % H₂. The syngas may also contain N₂ and CH₄ as well as trace components such as H₂S and COS, NH₃ and HCN. Other sources of the gas substrate include gases generated during petroleum and petrochemical processing and from steel mill operations. These gases may have substantially different compositions than typical syngas, and may be essentially pure hydrogen or essentially pure carbon monoxide. The gas substrate may be obtained directly from gasification or from petroleum and petrochemical processing or may be obtained by blending two or more streams.

The gas substrate may be treated to remove or alter the composition including, but not limited to, removing components by chemical or physical sorption, membrane separation, and selective reaction. Components may be added to the gas substrate such as nitrogen or adjuvant gases such as ammonia and hydrogen sulfide. See copending U.S. patent application Ser. Nos. 13/304,902, referenced above, and 13/440,953, filed Apr. 5, 2012, hereby incorporated by reference in its entirety.

### Microorganisms

The oxygenated organic compound produced in the processes of this invention will depend upon the microorganism used for the fermentation and the conditions of the fermentation. Bioconversions of CO and H₂/CO₂ to acetic acid, n- butanol, butyric acid, ethanol and other products are well known. For example, a concise description of biochemical pathways and energetics of such bioconversions has been presented by Das, A. and L. G. Ljungdahl, Electron Transport System in Acetogens and by Drake, H. L. and K. Kusel, Diverse Physiologic Potential of Acetogens, appearing respectively as Chapters 14 and 13 of Biochemistry and Physiology of Anaerobic Bacteria, L. G. Ljungdahl eds,. Springer (2003). Any suitable microorganisms that have the ability to convert the syngas components: CO, H₂, CO₂ individually or in combination with each other or with other components that are typically present in syngas may be utilized. Suitable microorganisms and/or growth conditions may include those disclosed in U.S. patent application Ser. No. 11/441,392, filed May 25, 2006, entitled “Indirect Or Direct Fermentation of Biomass to Fuel Alcohol,” which discloses a biologically pure culture of the microorganism Clostridium carboxidivorans having all of the identifying characteristics of ATCC no. BAA-624; U.S. Pat. No. 7,704,723 entitled “Isolation and Characterization of Novel Clostridial Species,” which discloses a biologically pure culture of the microorganism Clostridium ragsdalei having all of the identifying characteristics of ATCC No. BAA-622; both of which are incorporated herein by reference in their entirety. Clostridium carboxidivorans may be used, for example, to ferment syngas to ethanol and/or n-butanol. Clostridium ragsdalei may be used, for example, to ferment syngas to ethanol.

Suitable microorganisms and growth conditions include the anaerobic bacteria Butyribacterium methylotrophicum, having the identifying characteristics of ATCC 33266 which can be adapted to CO and used and this will enable the production of n-butanol as well as butyric acid as taught in the references: “Evidence for Production of n-Butanol from Carbon Monoxide by Butyribacterium methylotrophicum,” Journal of Fermentation and Bioengineering, vol. 72, 1991, p. 58-60; “Production of butanol and ethanol from synthesis gas via fermentation,” FUEL, vol. 70, May 1991, p. 615-619. Other suitable microorganisms include: Clostridium Ljungdahlii, with strains having the identifying characteristics of ATCC 49587 (U.S. Pat. No. 5,173,429) and ATCC 55988 and 55989 (U.S. Pat. No. 6,136,577) that will enable the production of ethanol as well as acetic acid; Clostridium autoethanogemum sp. nov., an anaerobic bacterium that produces ethanol from carbon monoxide. Jamal Abrini, Henry Naveau, Edomond-Jacques Nyns, Arch Microbiol., 1994, 345-351; Archives of Microbiology 1994, 161: 345-351; and Clostridium Coskatii having the identifying characteristics of ATCC No. PTA—10522. U.S. Pat. No. 8,143,037. All of these references are incorporated herein in their entirety.

### Fermentation Conditions and Bioreactors

The processes of this invention involve the use of a fermentation broth containing freely suspended microorganisms. The fermentation broth will comprise an aqueous suspension of microorganisms and various media supplements. Suitable microorganisms generally live and grow under anaerobic conditions, meaning that dissolved oxygen is essentially absent from the fermentation liquid. The various adjuvants to the aqueous fermentation broth may comprise buffering agents, trace metals, vitamins, salts etc. Adjustments in the fermentation broth may induce different conditions at different times such as growth and non-growth conditions which will affect the productivity of the microorganisms. U.S. Pat. No. 7,704,723, hereby incorporated by reference in its entirety, discloses the conditions and contents of suitable fermentation broths for bioconversion CO and H₂/CO₂ using anaerobic microorganisms.

Anaerobic fermentation conditions include a suitable temperature, say, between 25° and 60° C., frequently in the range of about 30° to 40° C. The conditions of fermentation, including the density of microorganisms, fermentation broth composition, and syngas residence time, are preferably sufficient to achieve the sought conversion efficiency of hydrogen and carbon monoxide and will vary depending upon the design of the fermentation reactor and its operation. The pressure above the fermentation broth may be subatmospheric, atmospheric or super atmospheric, and is usually in the range of from about 90 to 1000 KPa absolute and in some instances higher pressures may be desirable for biofilm fermentation reactors. As most reactor designs, especially for commercial scale operations, provide for a significant height of the fermentation broth, the pressure will vary within the fermentation reactor based upon the static head.

The fermentation conditions are preferably sufficient to result in at least about 40 or 50 percent conversion of the carbon monoxide in gas feed. For commercial operations, the fermentation operation preferably provides a total molar conversion of hydrogen and carbon monoxide in the net gas feed in the range of about 85 to 95 percent. Due to the low solubilities of carbon monoxide and hydrogen in the aqueous phase, achieving these high conversions may require one or more of using multiple fermentation reactors and recycling off gas from a reactor.

The rate of supply of the gas feed under steady state conditions to a fermentation reactor is such that the rate of transfer of carbon monoxide and hydrogen to the liquid phase matches the rate that carbon monoxide and hydrogen are bioconverted. The rate at which carbon monoxide and hydrogen can be consumed will be affected by the nature of the microorganism, the concentration of the microorganism in the fermentation broth and the fermentation conditions. As the rate of transfer of carbon monoxide and hydrogen to the fermentation broth is a parameter for operation, conditions affecting the rate of transfer such as interfacial surface area between the gas and liquid phases and driving forces are important.

To increase the conversion of carbon monoxide and hydrogen in the fresh gas feed, off-gas withdrawn from a fermentation reactor may be recycled or passed to a fermentation reactor that is sequential in gas feed flow. See, for instance, U.S. Published Patent Applications Nos. 2012/0003707 A1, and 2013/003706, both hereby incorporated by reference in their entireties. Where off-gas is recycled, the portion of off-gas recycled is generally selected to avoid an undue build-up of the concentration of inerts and other gases in the fermentation reactor.

The fermentation reactors used in this invention may be of any suitable design employing free suspensions of microorganisms in a fermentation broth; however, preferably the design and operation provides for a high conversion of carbon monoxide and hydrogen to oxygenated organic compound. Fermentation reactors include, but are not limited to, bubble column reactors; jet loop reactors; stirred tank reactors; trickle bed reactors; biofilm reactors; moving bed reactors; and static mixer reactors including, but not limited to, pipe reactors. See, for instance, United States Published Patent Applications Nos. 2013/0078687 A1, 2013/0078688 A1 and 2013/0078689 A1, all hereby incorporated by reference in their entireties.

### Withdrawal of Fermentation Broth from the Bioreactor and Processing

A portion of the fermentation broth is withdrawn intermittently or continuously from the bioreactor for product recovery. The rate of withdrawal of the fermentation broth is typically sufficient to not to exceed a desired maximum concentration of oxygenated organic compound in the bioreactor. As the oxygenated organic compound can be toxic to the microorganisms, the maximum concentration is usually selected to assure that the microorganisms in the fermentation broth are not unduly adversely affected. The toxic concentration of an oxygenated organic compound depends upon the oxygenated organic compound (e.g., butanol is more toxic than ethanol) and the microorganism. Generally, higher concentrations of oxygenated organic compound in the fermentation broth are preferred to facilitate and reduce costs of product separation, e.g., by distillation.

The withdrawal of the fermentation broth from the bioreactor may occur at one or more locations in the bioreactor. Preferably the withdrawal is made at a point where the gas phase substrate is not also withdrawn from the bioreactor, and more preferably for deep tank reactors, especially bubble column reactors, where a lower concentration of dissolved substrate exists. Preferably the withdrawn fermentation broth contains at least some substrate, for instance at least about 0.5 milligram per liter, say about 1 to 10 or more milligrams per liter, of at least one of hydrogen and carbon monoxide. The contained substrate allows time for processing of the withdrawn fermentation broth prior to the onset of starvation of the microorganisms contained therein.

At least an aliquot portion of the withdrawn fermentation broth is continuously or intermittently passed to a solids separating unit operation to provide a solids depleted liquor and a solids rich stream. The portion passed to the solids separating unit operation is at least sufficient to provide the sought amount of microbial concentrate in storage, and if desired, the entire withdrawn fermentation broth can be passed to the solids separation unit operation. The remaining portion can be directed to product recovery or used for other purposes such as providing motive fluid for any injectors used to introduce feed gas into the bioreactor such as disclosed in U.S. Published Patent Applications Nos. 2013/0078687 A1 and 2013/0078689 A1. In any event, sufficient fermentation broth needs to be removed from the bioreactor for operational purposes as discussed above.

Any suitable solids separating unit operation may be used. Preferred unit operations are those using centrifugal forces such as disk stack centrifuges, decanter centrifuges, tubular and chamber bowl centrifuges, and imperforate basket centrifuges. While often microorganisms are capable of withstanding the forces of the solids separating unit operations, unit operations that provide sought concentrations of solids with less than about 20, preferably less than about 10, percent cell rupturing are preferred. The solids separation unit operation is preferably adapted to relatively quickly effect the separation from the time that the fermentation broth is withdrawn from the bioreactor in order to reduce mortality due to consumption of substrate and starvation of the microorganisms. Often the duration of time between the withdrawal of the fermentation broth to completion of the separation is less than about 2 hours, preferably less than about 1 hour. The temperature of the fermentation broth passed to the solids separating unit operation may vary over a wide range. Generally the fermentation broth is provided without any significant cooling, e.g., in the range of about 30° C. to 40 ° C.

The solids separating unit operation typically provides a solids rich stream that is at least about 5, preferably at least about 20, say, between about 50 to 200 or more, times more concentrated in solids than the withdrawn fermentation broth. As the solids depleted liquor contains the sought oxygenated organic compound, higher product recoveries can be obtained where the solids separating unit provides higher solids concentrations in the solids rich stream. A balance exists in commercial operations between the incremental recovery of oxygenated organic compound and the capital and operating costs to achieve the incremental recovery. For typical centrifuging, the solids concentration in the solids rich stream is generally in the range of about 50 to 125 or 200 grams per liter. As not only oxygenated organic compound but also substrate and other nutrients as well as other metabolites from the fermentation are contained in the liquid phase, the solids separating unit operation inherently reduces the mass ratio of these components to solids.

The solids depleted liquor is directed to product recovery. Product recovery can consist of known equipment arrangements for recovery of liquid products from the fermentation broth including, but not limited to, distillation columns, membrane systems and other separation equipment. US 2009/0215139 A1 shows an arrangement for a product recovery reactor that recovers an ethanol product from a bioreactor, herein incorporated by reference in its entirely.

At least a portion of the solids rich stream from the solids separating unit operation provides microorganisms for storage. In one embodiment, a portion of the solids rich stream is passed back to the bioreactor. A preferred option is to add aqueous fluid to the solids rich stream which can serve as make-up water for the fermentation broth and can provide some cooling to dissipate the build-up of temperature in the bioreactor due to the heat of fermentation. The aqueous fluid may contain nutrients and additives for the fermentation broth. Where a portion of the solids rich stream is returned to the bioreactor, the relative volume with respect to the portion used for collecting microorganisms for storage can vary widely from returning 100 percent to the bioreactor at times when no need exists for microorganisms for storage to none at times when all microorganisms are sought for storage. Where the portion of the solids rich stream is used for cooling the fermentation broth in the bioreactor, the cooling requirements will be a significant factor in determining the relative portion returned to the bioreactor. As stated above, a portion of the fermentation broth may by-pass the solids separation unit operation and be directed to product recovery. Where this embodiment is used, the operator of the facility has a plurality of options to maintain a desired cell retention time in the bioreactor and to obtain a sought amount of microorganism for storage.

The solids rich stream, before or after obtaining an aliquot portion for recycle to the bioreactor, is admixed with a cooler, aqueous fluid (diluting water stream) to provide a diluted solids stream, or combined stream. As stated above, the solids separating unit operation has removed substrate and other nutrients available to the microorganisms. Accordingly, the processes of this invention admix a cooler, diluting water stream with the solids rich stream and the diluting water stream to enable the temperature of the solids rich stream to be quickly reduced, thereby reducing the metabolic activity of the microorganisms without undue potential for physical stress to the microorganisms. In most instances, the amount of diluting water stream provided is sufficient to provide a combined stream having a reduced temperature, say, below about 30° C., say, between about 10° C. and 25° C., and most preferably between about 10° C. and 20° C. The amount of diluting water stream will, in part, depend upon the temperature of the diluting water stream and the sought temperature of the combined stream. Frequently, the temperature of the diluting water stream is below about 25° C., and sometimes below about 20° C., or even below about 15° C., say, between about 0° C. to 15° C. Often, sufficient diluting water stream is provided to increase the volume of the combined stream as compared to the solids rich stream by at least about 20 volume percent, say, between about 20 and 500, volume percent. Although higher dilutions can be used, they tend to be less preferred due to the energy required from reconcentrating the combined stream. The amount of diluting water stream may also be dependent upon other sought functions of the diluting water stream.

For instance, the diluting water stream can serve to reduce the concentration of the oxygenated organic compound remaining from that in the solids rich stream as well as other metabolites and components that may adversely affect the microorganisms, especially during reactivation.

Most often, the diluting water stream is used to provide components to enhance the survival and viability of the microorganisms during their storage and reactivation. The removal of most, if not essentially all, of the dissolved substrate and other nutrients as well as oxygenated organic compound and other metabolites, especially acetate ion in the case of acetogenic fermentation, by the solids separating unit operation enables the composition of the aqueous environment to be reconstituted. In this respect, the diluting water stream contains one or more organic carbon food source and other nutrients.

The carbon food source may be and suitable carbon food source with the proviso that the concentration of the food source in the diluting water stream and in the microorganism concentrate not be so great as to adversely affect the microorganisms. Hence, while a food source such as ethanol, propanol or butanol can be used, a practical limit exists as to the mass of such food source that can be provided per unit mass of solids. Therefore, to achieve a desired mass of food source per unit mass of solids, it may be necessary to limit the degree of concentration of the subsequent solids separation unit operation. Most often, whether or not a food source that can be toxic in higher concentrations, such as ethanol, propanol or butanol, is used, the food source comprises a substantially non-toxic carbohydrate such as a sugar-based moiety, e.g., of 5 to 12 carbons, and/or an oxygenated organic substrate. Examples of oxygenated organic substrates include citrate, glyoxylate, ketoglutarate, succinate, oxaloacetate, glutamate, malate, vanillin, vanillate, aromatic methyl ethers, carboxylated aromatics, and pyruvate. Examples of sugar-based moieties include, but are not limited to, monosaccharides and disaccharides such as xylose, xylitol, galactose, glucose, mannose, fructose, sorbitol, lactose, sucrose and maltose, and preferably the sugar-based moieties are monosaccharides. Of the sugar-based moieties, those for which the microorganisms have a moderate to limited consumption rate include xylose, mannose, sucrose, xylitol and sorbitol. Fructose and sucrose are sugar-based moieties more rapidly metabolized by the microorganisms. The low metabolic activity retards the consumption of sugar-based moieties such that sustenance rather than population growth is fostered.

In addition or alternatively, the diluting water stream may contain one or more of a nitrogen source, sulfur source (especially cysteine), and micronutrients. U.S. Pat. No. 7,704,723 discussed above describes various nutrients and additives that may be suitable for inclusion in the diluting water stream.

In one aspect of the invention, a plurality of carbon food sources is used that differ in the rate at which the microorganisms metabolize the compounds and, optionally, the lag time for the microorganisms to metabolize the compounds. For instance, the lag time for citrate can be substantially longer, e.g., up to 3 days or more. Although the metabolic response of various organisms can vary, the general principles can be used to provide a long usable carbon source for the microorganisms in storage.

The concentration of carbon food source in the diluting water stream is such that a desired ratio of food source to solids is achieved after the solids separation unit operation to provide the sought microorganism concentrate. The concentration of carbon food source will depend, among other things, upon the concentration of solids in the microorganism concentrate, the nature of the microorganism, the temperature and metabolic activity of the microorganism in the microorganism concentrate during storage, and the expected duration of the storage. As each of these variables can be selected over a wide range, the concentration of the carbon food source in the diluting water stream will similarly reside within a broad range. As can be readily appreciated, higher concentrations of carbon food source in the diluting water stream will be required as the concentration of solids in the microorganism concentrate increases. Similarly, where the temperature of storage and the microorganisms have greater metabolic activity and where longer storage times are anticipated, higher concentration of food source in the diluting water stream will be used. Using these general principles, the artisan can determine the optimal concentrations of carbon food source in the diluting water stream for a given situation. In some instances, the optimal concentrations of carbon food source in the diluting water stream can be ascertained by measuring the amount of carbon food source in the microorganism concentrate after storage, and then adjusting the concentration of food source in the diluting water stream such that subsequently produced microorganism concentrate has the sought concentration of carbon food source in the microorganism concentrate after storage. In general, the mass ratio of carbon food source to solids in the microorganism concentrate after storage is in the range of about 0.0001:1 to 0.1:1. In some instances the mass ratio of carbon food source to solids in the microorganism concentrate is in the range of 1:100 or 5:100 to 20:100 although higher ratios of carbon food source to solids can be used.

In addition or alternatively, the diluting water stream may contain one or more of a nitrogen source, sulfur source (especially cysteine), and micronutrients. Various adjuvants to the microorganism concentrate may comprise buffering agents, trace metals, vitamins, salts etc. U.S. Pat. No. 7,704,723 discussed above describes various nutrients and additives that may be suitable for inclusion in the diluting water stream. Since the combined stream is subjected to the solids separation unit operation to provide the microorganism concentrate, the concentration of these nutrients and additives in the diluting water stream needs to reflect the removal of liquid during solids separation. The preferred microorganism concentrates of this invention contain cysteine which serves both as a sulfur source and a reductant in the event of oxygen incursion into the microorganism concentrate. It can also provide nitrogen nutrient. The amount of cysteine used will depend, in part, upon the amount of carbon food source provided, and thus its concentration can vary over a wide range. However, in many instances, cysteine is provided in an amount of at least about 0.05, say, between about 0.1 to 5 or 10, grams (anhydrous) per 100 grams of solids. Ferrous ion is also another preferred component of the microorganism composite and is often present in the amount of at least about 1, say, between about 3 and 100 or more, milligrams per 100 grams of solids in the microorganism concentrate. Pantothenate ion is yet another preferred component of the microorganism concentrate and is often in an amount of at least about 0.05, say, between about 0.1 and 2.5 or 3, milligrams per 100 grams of solids in the microorganism concentrate.

In some instances, nutrients and other additives for the microorganism concentrate can be provided by suitable additive packages, e.g., yeast extract.

The combined stream is subjected to a solids separation unit operation to provide a highly concentrated, microorganism-containing fluid (microorganism concentrate). This unit operation also provides a solids depleted water stream that can be used elsewhere in the process, be cooled and recycled, sent to product recovery or be disposed. Any suitable solids separating unit operation may be used. Preferred unit operations are those using centrifugal forces such as disk stack centrifuges, decanter centrifuges, tubular and chamber bowl centrifuges, and imperforate basket centrifuges. While often microorganisms are capable of withstanding the forces of the solids separating unit operations, unit operations that provide sought concentrations of solids with less than about 20, preferably less than about 10, percent cell rupturing are preferred. The duration of time between the withdrawal of the fermentation broth to the formation of the microorganism concentrate is often less than about 3 hours, preferably less than about 1 hour, although with lower combined stream temperatures, the duration may be able to be extended without undue adverse effects on the microorganism population.

The microorganism concentrate contains at least about 30, preferably at least about 50, grams per liter, and in some instances up to about 500 or more, and most often between about 70 and 200, grams per liter. As the solids concentration in the microorganism concentrate increases, so does the viscosity. The microorganism concentrate can thus be essentially a solid; however, in most instances the microorganism concentrate is in the form of a flowable slurry to facilitate handling. In one preferred embodiment the microorganism concentrate has a sufficient microorganism density that the microorganisms tend to reduce their metabolic rates as compared to a cell density of 1 gram per liter. In some instances, the metabolic rate is reduced to below about 50 percent, say, below about 10, and sometimes, below about 5, percent of that at a density of 1 gram per liter, all else being maintained the same. In another preferred embodiment, the solids density is sufficient to provide at the temperature of the microorganism concentrate a dynamic viscosity of at least about 1 Pascal second (1000 centipoise) but flowable. In some instances, the viscosity is in the range of about 2 to 200, say, 5 to 100, Pascal seconds. At this density, the microorganism concentrate is capable of flowing for transport. In other embodiments, the viscosity is such that during storage, in the absence of external forces, little or no mixing of the microorganism concentrate occurs such that the stored microorganisms can be maintained at a given average storage time. Moreover, the high viscosity of the concentrate can protect a significant percentage of the viable microorganisms from adventitious oxygen, e.g., from leaks or handling.

### Storage of the Microorganism Concentrate

The microorganism concentrate is stored until needed without the addition of syngas. Storage includes transport. Preferably the storage is under static conditions. The storage preferably occurs at temperatures below about 25° C., but above the temperatures at which the concentrate freezes, preferably between about 0° C. to 20° C. Typically at higher storage temperatures the viable lifetime of the concentrate is less than that at lower temperatures. Nevertheless, especially with concentrates having higher solids concentrations, e.g., at least about 80, preferably at least about 250, grams per liter, storage at between about 10° C. to 20° C. or 25° C., can exceed 5 or 10 days without undue adverse effect on the population of microorganisms. In some instances, the temperature of the storage is between about 1° C. and 10° C. or 15° C. The pH for the storage may vary widely, and is usually in the range of about 4.5 to 7, preferably about 5.0 to 5.8 or 6. Buffers may be used to assist in maintaining the sought pH. However, in many instances the reduced metabolic activity of the microorganisms in the microorganism composite is sufficiently low that undue reduction of pH during storage is not observed even in the absence of buffers.

The duration of the storage to maintain a viable microorganism concentrate, e.g., with at least about 20, preferably at least about 50, percent of the cells placed into storage, being viable, will depend upon the temperature of the storage and the nutrients and additives provided to the microorganism concentrate prior to storage. Often, the storage temperature is in the range of about 0° C. to 25° C., say, 2° C. or 4° C. to 20° C. In some preferred processes of this invention, the microorganism concentrate can be stored for extended durations, e.g., for at least about 3 days, and in some instances in excess of about 30 days, at about 10° C. to 20° C., which reduces costs for cooling. For instance, cooling tower water may be adequate to maintain suitable temperatures. In any event, the substantially reduced volume of the microorganism concentrate facilitates its storage in refrigerated facilities.

In preferred embodiments of this invention, the microorganism concentrate in storage is maintained at a desired average residence time by continuously or intermittently removing the oldest concentrate. This maintenance can be done in any suitable manner. Where the concentration has a high viscosity, the storage vessel may be adapted to remove the first in concentrate which would be at the distal end of the vessel since little intermixing of the concentrate will occur. Alternatively a plurality of vessels can be used with the oldest being purged to maintain the sought average residence time. Usually the average residence time of the microorganism concentrate in storage is between about 2 or 3 and 100, preferably 5 and 30, days. The long useful life of the microorganism concentrate in most instances enables a normal operation of the bioreactor including average microorganism life time in the bioreactor. Often the average microorganism life time in the bioreactor is between about 2 and 7 days.

In some instances carbon dioxide is provided in the microorganism concentrate. The carbon dioxide can be introduced directly to the microorganism concentrate or be added into the combined stream. Alternatively, the concentrate can be stored with a carbon dioxide rich gas in the head space. The concentration of carbon dioxide in the microorganism concentrate is preferably in an amount sufficient to modulate the consumption of any organic carbon source in the concentrate. Preferably sufficient carbon dioxide is present that the partial pressure of carbon dioxide in the head space is at least about 10, say, at least about 20, kilopascals. The balance of the headspace may be an inert gas such as nitrogen or methane. In some instances carbonate or bicarbonate anion is provided in the microorganism concentrate, e.g., as carbonic acid or salts of carbonate or bicarbonate anion, especially salts having some solubility in water such as ammonium, alkali metal, alkaline earth metal salts. The concentration of carbonate or bicarbonate anion in the microorganism concentrate is preferably in an amount sufficient to modulate the consumption of any organic carbon source in the concentrate.

### Reactivation

The stored, microorganism concentrate may be reactivated in any suitable manner which provides for the distribution of the microorganism concentrate to reconstitute the fermentation broth and increases the temperature of the concentrate to fermentation conditions. A bioreactor can be restarted using any suitable process. Where the start-up of the bioreactor involves increasing the population of the microorganisms and the volume of fermentation broth is increased as the population of the microorganisms increases, a preferred start-up process is disclosed in U.S. Published Patent Application No. 2013/0078693 A1, hereby incorporated by reference in its entirety. The microorganism concentrate may also be used to replenish a depleted population of microorganisms in a reactor. The depleted population in the bioreactor may have occurred due to an operational upset.

In preferred embodiments of this invention, the pH of the microorganism concentrate (or diluted microorganism concentrate) supplied to the bioreactor is in the range of about 4.5 to 7.5, more preferably between about 5 and 6.5, say, 5.3 to 5.8 or 6. Usually, the microorganisms are more quickly reactivated where the concentrate is at a higher pH than that of the fermentation broth at the desired steady state operation.

Preferably the temperature of the microorganism concentrate is gradually increased prior to being introduced into the bioreactor to avoid any undue stress on the microorganisms. The rate of increase is usually in the range of 0.25° C. to 5° C. per minute and the temperature of the microorganism concentrate supplied to the bioreactor is within about 10° C., preferably within about 5° C., of the temperature of the fermentation broth in the bioreactor. The temperature increase from the temperature of the storage may be effected in any suitable manner including indirect heat exchange or by direct heat exchange by dilution with a warm aqueous fluid, e.g., at a temperature of about 25° C. to 40° C.

### DRAWING

A general understanding of the invention and its application may be facilitated by reference to the drawing.

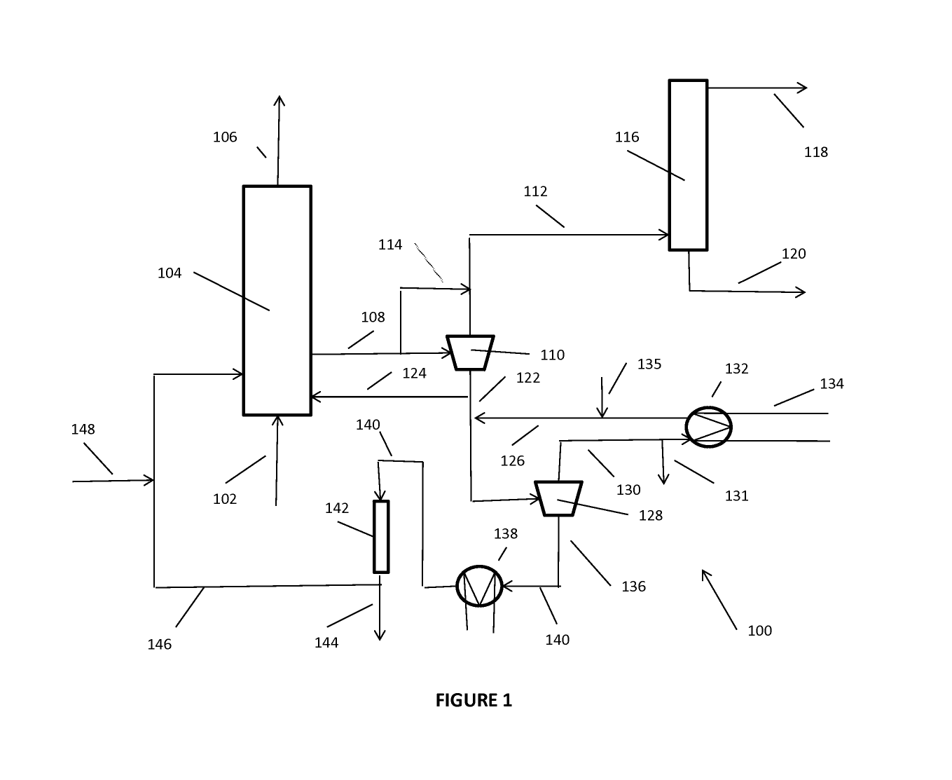

FIG. 1 is a schematic depiction of an apparatus generally designated as 100 suitable for practicing the processes of this invention. FIG. 1 omits minor equipment such as pumps, compressors, valves, instruments and other devices the placement of which and operation thereof are well known to those practiced in chemical engineering. FIG. 1 also omits ancillary unit operations. The process and operation of FIG. 1 will be described in the context of the recovery and production of ethanol. The process is readily adaptable to anaerobic fermentation reactors to make other oxygenated products such as acetic acid, butanol, propanol and acetone.

Syngas is provided by line 102 to the bottom of a deep, bubble column reactor 104 containing aqueous medium having a suspension of microorganism for the bioconversion of carbon monoxide and hydrogen to ethanol. Reactor 104 can be of any other suitable design including, but not limited to, stirred tank reactor, jet loop reactor, trickle bed reactor, and stirred tank reactor. More than one reactor may be used, and the reactors may be in parallel or series. The syngas may be of any suitable composition containing carbon monoxide and optionally hydrogen. The syngas is preferably treated to remove deleterious amounts of components that can adversely affect the microorganism for the bioconversion. The aqueous medium in reactor 104 is maintained under bioconversion conditions including temperature and the presence of nutrients and additives.

Off gases, which typically contain carbon monoxide, carbon dioxide and hydrogen are withdrawn from reactor 104 via line 106. A portion of these off gases can be recycled to reactor 104, e.g., by admixing with fresh syngas in line 102.

Continuously or intermittently aqueous medium is withdrawn from reactor 104 via line 108. The withdrawn aqueous medium contains ethanol and microorganisms. The withdrawn aqueous medium is passed to centrifuge 110 to provide a solids-depleted liquor containing ethanol which exits via line 112 and a solids-rich stream that exits via line 122. More than one centrifuge may be used, and it is understood that other phase separation unit operations can be used instead of centrifuge 110 or in combination with centrifuge 110 such as hydrocyclones, filtrations, and the like. The solids depleted liquor in line 112 is directed to distillation assembly 116 for the recovery of ethanol which is withdrawn via line 118. A bottoms stream comprised of water is removed from distillation assembly 116 via line 120.

Returning to line 108, in some instances it may be desired to bypass or only send a portion of the aqueous medium withdrawn from reactor 104 to centrifuge 110. Line 114 is provided such that all or a portion of the aqueous medium can by-pass centrifuge 110. The portion of the aqueous medium passed to centrifuge 110 may be essentially all the withdrawn aqueous medium or may be only that amount required to provide the sought volume of microorganism concentrate. As shown, line 124 is provided to recycle all or a portion of the solids rich stream to reactor 104. Viable microorganisms are thus returned and facilitate maintaining a desired cell density in the aqueous medium as well as a desired average cell residence time in reactor 104. Any aqueous medium that by-passes centrifuge 110 via line 114 will be subjected to temperatures used for the distillation which typically result in the death of the microorganisms. Hence, the bottoms stream in line 120 would contain solid debris and no viable microorganisms for recycling to reactor 104.

A diluting water stream from line 126 is admixed with the solids rich stream in line 122. As shown, the admixing is subsequent to the take-off via line 124. However, all or a portion of the diluting stream can be added prior to the take-off via line 124. The diluting stream preferably is a chilling stream and thus reduces the temperature of the solids-rich stream. Where added prior to the take-off via line 124, the chilling stream may serve both to remove heat generated by the fermentation and to provide make-up water and soluble nutrients and additives to the aqueous medium in reactor 104. The chilling also quickly reduces the temperature of the solids-rich stream and can do so with minimal physical stress on the microorganisms. The diluting water can also serve to provide nutrients and other additives that facilitate the storage of the microorganism concentrate and reactivation of the microorganisms after storage.

The combined streams in line 122 are passed to centrifuge 128. More than one centrifuge may be used, and it is understood that other phase separation unit operations can be used instead of centrifuge 128 or in combination with centrifuge 128 such as hydrocyclones, filtrations, and the like. Centrifuge 128 provides a solids depleted water stream that is passed via line 130 to indirect heat exchange cooler 132. Purge is removed from line 130 via line 131 to maintain a desired, steady state concentration of components in the diluting water stream. Cooler 132 uses cooling tower water provided by line 134 as the heat exchange fluid. The cooled diluting stream exits cooler 132 via line 126. Line 135 provides make-up water and nutrients and other additives to the diluting stream.

Centrifuge 128 also provides a microorganism concentrate stream which is passed via line 136 to chiller 138. Chiller 138 reduces the temperature to a desired temperature, and the heat exchange fluid for indirect heat exchange, depending upon the sought temperature, may be water with an antifreeze component or a refrigerant. Line 140 carries the chilled microorganism to storage tank 142. Storage tank 142 is adapted to maintain the microorganism concentrate at a desired temperature for storage. While chiller 138 and storage tank 142 are depicted as separate devices, it is to be understood that they can be combined.

Storage tank 142 is adapted to provide a plug flow of microorganism concentrate such that the first in concentrate is the first removed from storage tank 142. The viscous microorganism concentrate tends not to circulate in storage tank 142 and thus a tall, narrow vessel can be used as storage tank 142. Microorganism concentrate is removed from storage tank 142 via line 144. The removed microorganism concentrate may be shipped for use at another site or may be purged to maintain a sought average residence time of the microorganism concentrate in storage tank 142. The microorganism concentrate can be used to replenish microorganisms in a reactor or as a charge of microorganism to a reactor for start-up. As shown, the microorganism concentrate can pass via line 146 to a reactor for start-up. Line 148 provides a make-up water stream for admixing with the microorganism concentrate. The make-up water provides water to form the aqueous medium in reactor 102 and can serve to warm the microorganisms to facilitate bringing them to the desired operating temperature. The make-up water stream may also provide nutrients and additives to facilitate reactivation of the microorganisms.

It is claimed:

1. An integrated process for the anaerobic bioconversion of a gas feed containing gas substrate comprising at least one of carbon monoxide and of carbon dioxide and hydrogen in a fermentation broth containing a suspension of microorganisms suitable for converting said gas substrate to oxygenated organic compound and for providing a microorganism concentrate comprising:
a. contacting the gas feed with the fermentation broth under fermentation conditions in a bioreactor to bioconvert at least a portion of the gas substrate to oxygenated organic compound;
b. continuously or intermittently withdrawing from the bioreactor a portion of the fermentation broth to maintain the concentration of the oxygenated organic compound below that which would unduly adversely affect the microorganisms, said withdrawn portion containing microorganisms;
c. separating at least an aliquot portion of the withdrawn fermentation broth to provide a solids depleted liquor and a solids rich stream;
d. recovering a product comprising said oxygenated organic solvent from the solids depleted liquor;
e. diluting at least a portion of the solids rich stream with a cool, diluting water stream to provide a combined stream having a temperature below about 25° C. sufficient to reduce the metabolic activity of the microorganisms;
f. separating the combined stream into a microorganism concentrate having a solids content of at least about 30 grams per liter and a solids depleted water stream;
g. storing said microorganism concentrate without the addition of syngas at a temperature below about 25° C.

2. The process of claim 1 wherein the diluting water stream is at a lower temperature than that of the solids rich stream whereby the solids rich stream is cooled by direct heat exchange.

3. The process of claim 1 wherein the diluting water stream contains nutrients for the microorganism.

4. The process of claim 3 wherein the nutrients comprise carbon source.

5. The process of claim 4 wherein the nutrients comprise citrate.

6. The process of claim 4 wherein the nutrients comprise cysteine.

7. The process of claim 4 wherein the nutrients comprise ferrous ion and pantothenate ion.

8. The process of claim 1 wherein the microorganism concentrate contains at least about 70 grams of solid per liter.

8. The process of claim 1 wherein the microorganism concentrate is stored in the presence of carbon dioxide.

9. The process of claim 1 wherein the pH of the microorganism concentrate during storage is between about 4.5 and 7.

10. The process of claim 1 wherein the microorganism concentrate is further cooled after step (f).

11. The process of claim 1 wherein the oxygenated organic compound is at least one of ethanol, acetate, propanol, propionate, butanol and butyrate.

12. The process of claim 1 wherein the microorganism concentrate is reactivated, said reactivation comprising warming the microorganism concentrate to a temperature between about 25° C. and 40° C. and providing the microorganism concentrate in a fermentation broth.

13. The process of claim 12 wherein the microorganism concentrate contains nutrients during the warming.

14. In a process for providing a microorganism concentrate from a fermentation broth containing a free suspension of said microorganism from an anaerobic bioconversion of substrate comprising at least one of carbon monoxide and of carbon dioxide and hydrogen to oxygenated organic compound by separation to provide a solids depleted stream and a microorganism concentrate, the improvement comprising providing a concentrate containing at least about 70 grams of solids per liter.

15. In a process for storing a microorganism concentrate of microorganisms for anaerobic bioconversion of substrate comprising at least one of carbon monoxide and of carbon dioxide and hydrogen to oxygenated organic compound, the improvement comprising maintaining the viscosity of the microorganism concentrate sufficiently high to reduce intermixing of the concentrate and thereby enable a first in, first out inventory of concentrate.
